(19) United States
(12) Patent Application Publication
(10) Pub. No.: US 2025/0222441 A1
(43) Pub. Date: Jul. 10, 2025

(54) CATALYST AND PROCESS FOR THE DEPOLYMERIZATION OF POLYMERIC WASTE MATERIAL

(71) Applicant: Basell Poliolefine Italia S.R.L., Milano (IT)

(72) Inventors: Shahram Mihan, Bad Soden (DE); Volker Fraaije, Frankfurt (DE); Gerd Mannebach, Munstermaifeld (DE); Hans Leibold, Karlsruhe (DE); Frank Richter, St. Ingbert (DE)

(73) Assignee: Basell Poliolefine Italia S.R.L., Milano (IT)

(21) Appl. No.: 18/851,496

(22) PCT Filed: Mar. 30, 2023
(86) PCT No.: PCT/EP2023/058251

§ 371 Date: Sep. 26, 2024

(30) Foreign Application Priority Data
Mar. 30, 2022 (EP) 22165609.3

(51) Int. Cl. B01J 29/40; B01J 21/08; B01J 21/12; B01J 29/70; B01J 31/02; B01J 31/26; C07B 63/00; C08J 11/16; C10B 53/07; C10B 57/06

(52) U.S. Cl. CPC B01J 29/40; B01J 21/08; B01J 21/12; B01J 29/70; B01J 29/7007; B01J 31/02; B01J 31/26; C07B 63/00; C08J 11/16; C10B 53/07; C10B 57/06; C08J 2323/06; C08J 2323/12

(57) ABSTRACT

A catalyst for the depolymerization of polymeric waste material made from or containing an acidic component deposited on a solid support with the help of a coating agent as well as a depolymerization process using the catalyst. A process for the depolymerization of polymeric waste material, comprising: (a) providing a feedstock of polymeric waste material; (b) mixing the feedstock with a depolymerization catalyst of, thereby forming a mixture; (c) pyrolyzing the mixture, thereby generating gaseous fractions; (d) collecting the gaseous fractions; and (e) separating the collected gaseous fraction, thereby obtaining a gaseous and a liquid depolymerization product.

### CROSS-REFERENCE TO RELATED APPLICATIONS

Not applicable.

### FIELD OF THE DISCLOSURE

In general, the present disclosure relates to the field of chemistry. More specifically, the present disclosure relates to polymer chemistry. In particular, the present disclosure relates to a catalyst for the depolymerization of polymeric waste materials and depolymerization processes using the catalyst.

### BACKGROUND OF THE DISCLOSURE

Plastics include a wide range of synthetic and semi-synthetic materials that use polymers as their main ingredient. Their plasticity makes it possible for plastics to be molded, extruded or pressed into solid objects of various shapes. This adaptability, in combination with a wide range of other properties, such as light weight, durability and low production cost, has led to their widespread use. The production of plastics has increased dramatically over the last few decades. At the same time, the increasing amount of plastics has given rise to environmental concerns. In some instances, plastics are resistant to natural degradation processes. As such, the material may persist for centuries or longer, filling up landfill sites and even appearing in the food chain as microplastics.

Therefore, efforts are undertaken to improve the recycling of polymeric waste materials. In some instances, procedures of recycling rely on mechanical recycling and chemical recycling. For mechanical recycling, the plastics are mechanically transformed without changing their chemical structure, thereby permitting the plastics to be used to produce new materials. In some instances, mechanical recycling steps include collecting polymeric waste material, sorting the polymeric waste material into different types of plastic and colors, packaging the plastic waste material by pressing or milling, washing the polymeric waste material, drying the polymeric waste material and reprocessing the polymeric waste material into pellets by agglutinating, extruding and cooling the plastic, thereby obtaining recycled raw material for use in new articles.

During chemical recycling, the plastics are reprocessed. In some instances, the structure and chemical nature of the plastics are modified, such the plastics are usable as raw materials for different industries or as basic input or feedstock for manufacturing new polymeric products. In some instances, chemical recycling includes the steps of collecting plastic waste material and heating the plastic waste material, thereby yielding monomers from breaking down the polymers. In some instances, the resulting monomers are used to re-manufacture polymers or produce other synthetic chemicals.

In some instances, different types of polymeric waste material are collected together, thereby rendering chemical recycling troublesome. In some instances, the mixture of different polymers detrimentally affects control of the heating process, increases energy costs, and harms yields and the quality of the resulting products.

### SUMMARY OF THE DISCLOSURE

In a general embodiment, the present disclosure provides a catalyst for the depolymerization of polymeric waste material, made from or containing, as an active component, an acidic compound deposited on a particulate non-porous support with the aid of a coating agent.

In some embodiments, the particulate non-porous support is selected from the group consisting of sand, glass beads and metal particles.

In some embodiments, the acidic compound is selected from the group consisting of Al/Si mixed oxides, Al2O3, aluminosilicates, silica and zeolites.

In some embodiments, the coating agent is selected from the group consisting of oil, inorganic hydrogel, and combinations thereof.

In some embodiments, the catalyst is made from or containing 0.5 to 6.0 wt. %, alternatively from 1.0 to 4.0 wt. %, of the active component, based on the total weight of the catalyst.

In some embodiments, the present disclosure provides a process for the depolymerization of polymeric waste material, including the steps of: (a) providing a feedstock of polymeric waste material; (b) mixing the feedstock with the catalyst, thereby forming a mixture; (c) pyrolyzing the mixture, thereby generating gaseous fractions; (d) collecting the gaseous fractions; and (e) separating the collected gaseous fractions, thereby obtaining a gaseous and a liquid depolymerization product.

In some embodiments, the process further includes a step of (f) distilling the liquid depolymerization product.

In some embodiments, the polymeric waste material is made from or containing a plastic material selected from the group consisting of polyethylene (PE), polypropylene (PP), polystyrene (PS), polyethylene terephthalate (PET), polyvinylchloride (PVC), polyamide (PA), polyurethane (PU), polyacrylonitrile (PAN), polybutylene (PB), and mixtures thereof.

In some embodiments and after step (e), the depolymerization product is made from or containing more than 30 wt. %, alternatively more than 50 wt. %, alternatively more than 70 wt. %, of the gaseous product, based on the combined weight of the gaseous and liquid depolymerization product.

In some embodiments and after step (e), the gaseous depolymerization product is made from or containing at least 50 wt. %, alternatively at least 60 wt. %, alternatively at least 65 wt. %, of compounds of the formula CnH2n with n=2-4, based on the total weight of the gaseous depolymerization product.

In some embodiments, the liquid depolymerization product has a content of olefinic compounds, expressed as bromine number, (gram bromine per 100 grams of sample), of less than 150, alternatively from 10 to 100, alternatively from 15 to 80, alternatively from 20 to 70, alternatively from 25 to 100, determined according to ASTM D1159-01. In some embodiments, a 1H-NMR spectrum of the liquid depolymerization product shows less than 10 mol %, alternatively less than 5 mol %, alternatively no more than 3 mol %, of olefinic protons.

In some embodiments, the liquid depolymerization product has less than 10 mol %, alternatively less than 5 mol %, alternatively no more than 3 mol %, of aromatic components, measured as contents of aromatic protons in mol % as determined by 1H-NMR-spectroscopy.

In some embodiments, the polymeric waste material feedstock for depolymerization, has: (a) a polyolefin content of more than 50 wt. %, alternatively more than 60 wt. %, alternatively more than 70 wt. %, alternatively more than 80 wt. %, alternatively more than 90 wt. %, based on the total weight of the polymeric waste material feedstock; (b) a total ash content of less than 15 wt. %, alternatively less than 10 wt. %, alternatively less than 5 wt. %, alternatively less than 3 wt. %, determined as residue after heating the polymeric waste material feedstock at 800° C. for 120 hours in air; (c) a bulk density (i) from 70 to 500 g/l, alternatively from 100 to 450 g/l, wherein the polymeric waste material feedstock is in shredded form or (ii) from 300 to 700 g/l, wherein the polymeric waste material feedstock is in pellet form, determined according to DIN 53466; and (d) a content of total volatiles (TV) of less than 5%, alternatively less than 3%, measured as the weight loss of a 10 g sample at 100° C. after 2 hours at 200 mbar. In some embodiments, the polyolefin content is the total content of polypropylene (PP) and polyethylene (PE).

### BRIEF DESCRIPTION OF THE FIGURES

Certain embodiments of the disclosure will hereafter be described with reference to the accompanying drawings, wherein like reference numerals denote like elements. While the accompanying figures illustrate various implementations described herein, the accompanying figures do not limit the scope of the present disclosure.

FIG. 1 is a flowchart, depicting a process for depolymerization of plastic waste material.

FIG. 2 is a flowchart, depicting a process for depolymerization of plastic waste material.

### DETAILED DESCRIPTION OF THE DISCLOSURE

### Depolymerization Catalyst

In some embodiments, the present disclosure provides a catalyst for the depolymerization of polymeric waste material, made from or containing, as an active component, an acidic compound deposited on a particulate non-porous support with the aid of a coating agent.

In some embodiments, the catalyst provides for producing pure depolymerization products with gaseous fractions and low char generation. In some embodiments, the catalyst is separable from solid residue of the depolymerization process, thereby allowing for multiple uses of the catalyst.

Without being bound by a theory, it is believed that the catalyst supports the heat transfer generated during pyrolysis of the polymeric waste material.

In some embodiments and to assist in pyrolysis and depolymerization of the polymeric waste material, the catalyst is in particulate form. In some embodiments, particulate non-porous support is selected from the group consisting of sand, glass beads and metal particles. In some embodiments, the particulate non-porous support has any shape. In some embodiments, the shape is selected from the group consisting of spherical, cylindrical, and non-homogenous shapes. As used herein, the term “non-porous” refers to state of being not permeable to gases or liquids. In some embodiments, the gas is air. In some embodiments, the liquid is water. In some embodiments, the support has an average particle size D50 of 0.2 to 20 mm, alternatively 0.5 to 10 mm, alternatively from 1 to 8 mm, alternatively from 1 to 6 mm, determined according to sieve analysis in accordance with ISO 3310-1/ASTM E11. In some embodiments, the test sieve apparatus of Retsch with woven wire mesh sieves (Ø 125 mm-20 μm) is used as the sieving device.

In some embodiments, sand is the non-porous particulate support. In some embodiments, the sand has a particle size distribution of:

| | Min | Max |
|---|---|---|
| <3000 μm | 70% | 90% |
| >1400 μm | 40% | 80% |
| >1000 μm | 50% | 80% |
| <1000 μm | 0% | 20% |

In some embodiments, the acidic compound of the catalyst is selected from the group consisting of Al/Si mixed oxides, Al2O3, aluminosilicates, silica and zeolites. As used herein, the term “Al/Si mixed oxides” refers to a material made from or containing a mixture of Al2O3 and SiO2, having a neutral structure.

As used herein, the term “zeolites” refers to crystalline microporous aluminosilicates, which are built up from corner-sharing SiO4- and AlO4- tetrahedrons, having the structure Mn+x/n [AlO2]-x(SiO2)y]+ zH2O with n being the charge of the cation M and z defining the number of water molecules incorporated into the crystal structure. In some embodiments, M is an alkaline, alkaline earth metal, or hydrogen ion. In some embodiments, the ion is selected from the group consisting of H+, Na+, Ca2+, K+, and Mg2+. As described herein, zeolites differ from mixed Al/Si oxides by the zeolites' defined pore structure and ionic character. In some embodiments, the zeolite employed as the acidic compound is selected from the group consisting of Zeolite Y, Zeolite Beta, Zeolite A, Zeolite X, Zeolite L, and mixtures thereof, alternatively Zeolite Y and Zeolite Beta. In some embodiments, zeolites have the metal ion M substituted by a hydrogen. In some embodiments, the zeolite-type components are selected from the group consisting of ZSM-5, ZSM-11, ZSM-22, ZSM-23, ZSM-35, ZSM-48, ZSM-50, TS-1, TS-2, SSZ-46, MCM-22, MCM-49, FU-9, PSH-3, ITQ-1, EU-1, NU-10, silicalite-1, silicalite-2, boralite-C, boralite-D, BCA, and mixtures thereof.

In some embodiments, the acidic compound is an Al/Si mixed oxide. In some embodiments, the composition of the Al/Si mixed oxide is adjusted. In some embodiments, the acidic compound contains Al2O3 in an amount from 20 to 99 wt. %, alternatively from 30 to 80 wt. %, alternatively from 40 to 70 wt. %, based on the total weight of the acidic compound. In some embodiments, the acidic compound contains SiO2 in an amount from 1 to 80 wt. %, alternatively from 20 to 70 wt. %, alternatively from 30 to 60 wt. %, based on the total weight of the acidic compound.

In some embodiments, the acidic compound contains an excess of Al2O3 with respect to SiO2, thereby improving the depolymerization process. In some embodiments, the acidic compound is made from or containing an excess of Al2O3 with respect to SiO2. In some embodiments, the weight ratio of Al2O3 to SiO2 in the acidic compound is from 99:1 to 30:70, alternatively from 9:1 to 3:2, alternatively from 4:1 to 3:2.

In some embodiments, the determination of the SiO2 and Al2O3 content of the acidic compound is carried out by atomic emission spectroscopy using an inductively coupled plasma (ICP-AES).

In some embodiments, the coating agent employed in the catalyst is selected from the group consisting of oil, inorganic hydrogel, and combinations thereof. In some embodiments, the inorganic hydrogel is silica hydrogel. In some embodiments, the oil is aromatic-free white mineral oil. In some embodiments, the aromatic-free white mineral oil is based on iso-paraffins. In some embodiments, the oil has a kinematic viscosity at 20° C. of 140 to 180 mm²/s, alternatively 150 to 170 mm²/s. In some embodiments, the oil has a kinematic viscosity at 40° C. of 40 to 80 mm²/s, alternatively 50 to 70 mm²/s. In some embodiments, the oil has a kinematic viscosity at 100° C. of 5 to 15 mm²/s, alternatively 7 to 10 mm²/s. In some embodiments, the kinematic viscosity is determined according to ISO 3104.

In some embodiments, the amount of coating agent ranges from 1 to 300%, alternatively from 2 to 150% wt, alternatively from 5 to 100% wt, alternatively 10-80% wt, based on amount of acidic compound. For hydrogels, the amount to be employed is referred to the dry weight.

In some embodiments, the catalyst is made from or containing 0.5 to 6 wt. %, alternatively 2 to 4 wt. %, of the active compound, based on the total weight of the catalyst.

In some embodiments, the catalyst is obtained by mixing the particulate non-porous support and the coating agent, thereby obtaining a mixture, and then adding the acidic compound in the form of a powder to the resulting mixture. In some embodiments, the mixture is heat treated, thereby obtaining the catalyst. In some embodiments, the heat treatment is carried out at a temperature of 100 to 600° C. In some embodiments, the particulate non-porous support is subjected to a drying step before being mixed with the coating agent.

In some embodiments, the catalyst is obtained by a process including the steps of: (a) mixing the particulate non-porous support and the coating agent, thereby yielding a mixture; and (b) adding the acidic compound in powder form to the mixture of step (a).

In some embodiments, the catalyst is made from or containing: (a) sand as a particulate non-porous support; (b) an Al/Si mixed oxide or a zeolite as an acidic compound; and (c) mineral oil or silica hydrogel as a coating agent.

In some embodiments, the catalyst is reactivated by heating, thereby allowing multiple uses and conserving resources. In some embodiments, component (c) is a hydrogel. In some embodiments, the catalyst is re activable by heat treatment. In some embodiments, the heat treatment is carried out at a temperature of 500 to 900° C., alternatively 550 to 900° C., alternatively 600-850° C. In some embodiments, the heat treatment occurs in an oxidative atmosphere such as air or oxygen. In some embodiments, the treatment time is selected by experimental investigation for balancing catalyst activity and energy consumption. In some embodiments, carbon residue concentration on the catalyst is a parameter to consider. In some embodiments, carbon residue of regenerated catalyst is less than 20 weight % of the catalyst, alternatively less than 15%, alternatively less than 10%, alternatively less than 5%. In some embodiments, treatment times are in the range of 0.5 to 100 hours, alternatively 1 to 20 hours, alternatively 2 to 10 hours.

### Depolymerization

In some embodiments, the present disclosure provides a process for the depolymerization of plastic waste material, including the steps of: (a) providing a feedstock of plastic waste material; (b) mixing the feedstock with a catalyst, thereby forming a mixture; (c) pyrolyzing the mixture, thereby generating gaseous fractions; (d) collecting the gaseous fractions; and (e) separating the collected gaseous fractions, thereby obtaining a gaseous and a liquid depolymerization product.

In some embodiments, the catalyst is separated from the solid content, remaining after the depolymerization process, and reintroduced into the process.

In some embodiments, the collected gaseous fractions are separated into liquid and gaseous depolymerization products by condensation. In some embodiments, the liquid depolymerization product and the gaseous depolymerization product are further processed.

In some embodiments, the process generates little to no char. In some embodiments, the residue of the depolymerization process has a char content of less than 5 wt. %, alternatively less than 2 wt. %, based on the total weight of the depolymerization product.

In some embodiments, the liquid depolymerization product is further separated. In some embodiments, the process further includes a step of (f) distilling the liquid depolymerization product.

In some embodiments, the process yields a depolymerization product with a high gaseous content. In some embodiments, the gaseous content in the depolymerization product after step (e) is more than 30 wt. %, alternatively more than 50 wt. %, alternatively more than 70 wt. %, based on the combined weight of the gaseous and liquid depolymerization product.

In some embodiments, the gaseous fraction of the depolymerization product is made from or containing a high content of monomeric olefinic C2-C4-compounds. In some embodiments, the monomeric olefinic C2-C4-compounds are useful for further processing. In some embodiments, the monomeric olefinic C2-C4-compounds are useful for the production of polymers. In some embodiments, the gaseous depolymerization product are used directly as feedstock in cracking processes and subsequent polymerization. In some embodiments, the gaseous product is made from or containing light olefins and light alkanes and is transferred to a downstream cracker, bypassing the ovens and thereby producing polymerization grade monomer streams. In some embodiments, other side products are cracked in the oven. In some embodiments, the other side products are selected from the group consisting of ethane, propane, and butane. In some embodiments, the steps of treating the depolymerization product to obtain the monomers are bypassed, thereby saving energy and reducing CO2 output.

### Polymeric Waste Material Feedstock

In some embodiments, the process is for recycling a variety of polymeric waste materials, alternatively plastic waste materials. In some embodiments, the polymeric waste material feedstock is made from or containing polymeric materials, alternatively materials formed from synthetic polymers. In some embodiments, the polymeric materials are selected from the group consisting of polyolefins, polystyrene, polyethylene terephthalate (PET), polyvinyl chloride (PVC), polyamide, polycarbonate, polyurethane, polyester, natural and synthetic rubber, tires, filled polymers, composites and plastic alloys, and plastics dissolved in a solvent. In some embodiments, the polyolefins are selected from the group consisting of polyethylene and polypropylene. In some embodiments, other hydrocarbon materials are used as polymeric waste material feedstock. In some embodiments, the hydrocarbons are selected from the group consisting of biomass, bio oils, and petroleum oils. In some embodiments, the process is used with other hydrocarbons. In some embodiments and for the production of light gas olefins, a plastic feedstock is made from or containing polyolefins. In some embodiments, the feedstock is selected from a group consisting of a mixture of various different plastics and hydrocarbon materials.

In some embodiments, the polymeric waste material feedstock is made from or containing a single type of polymeric waste material, alternatively a mixture of two or more different polymeric waste materials. In some embodiments, the polymeric waste material feedstock is provided in a variety of different forms. In some embodiments and in small scale operations, the polymeric waste material feedstock is in the form of a powder. In some embodiments and in large scale operations, the polymeric waste material feedstock is in the form of pellets or in form of shredded flakes and/or small pieces of film. In some embodiments, the pellets have a particle size from 1 to 20 mm, alternatively from 2 to 10 mm, alternatively from 2 to 8 mm. In some embodiments, the shredded flakes or small pieces of film have a particle size from 1 to 20 mm. As used herein, a particles size in a defined range means that 90 wt. % of the particles have a particle size which is in the defined range. In some embodiments, particle size is determined by sieving or by using a Beckman Coulters LS13320 laser diffraction particle size analyzer.

In some embodiments, a polymeric waste material is made from or containing mostly plastic material. In some embodiments, a polymeric waste material is named after the type of polymer which forms the predominant component of the polymeric waste material. In some embodiments, a polymeric waste material employed as feedstock is made from or containing more than 25 wt. %, alternatively more than 40 wt. % alternatively more than 50 wt. %, of the polymeric material, based upon the total weight of the polymeric waste material. In some embodiments, the polymeric waste material feedstock is further made from or containing additives. In some embodiments, the additives are selected from the group consisting of fillers, reinforcing materials, processing aids, plasticizers, pigments, light stabilizers, lubricants, impact modifiers, antistatic agents, inks, and antioxidants.

In some embodiments, the polymeric waste materials are made from or containing polyolefins and polystyrene. In some embodiments, the polyolefins are selected from the group consisting of high-density polyethylene (HDPE), linear low-density polyethylene (LLDPE), low-density polyethylene (LDPE), ethylene-propylene-diene monomer (EPDM), and polypropylene (PP). In some embodiments, the polymeric waste materials are made from or containing a mixture of polyolefins and polystyrene.

In some embodiments, other polymeric waste materials are used in the depolymerization process. In some embodiments, the other polymeric waste materials are selected from the group consisting of polyvinyl chloride, polyvinylidene chloride, polyethylene terephthalate, polyurethane (PU), acrylonitrile-butadiene-styrene (ABS), nylon, and fluorinated polymers. In some embodiments, those polymers are present in an amount of less than 50 wt. %, alternatively less than 30 wt. %, alternatively less than 20 wt. %, alternatively less than 10 wt. %, of the total weight of the dry weight polymeric waste material feedstock.

In some embodiments, the polymeric waste material is made from or containing one or more thermoplastic polymers. In some embodiments, the polymeric waste material is free of thermosetting polymers. In some embodiments, the content of thermosetting polymers is less than 15 wt. %, alternatively less than 10 wt. %, alternatively less than 5 wt. %, of the polymeric waste material feedstock.

In some embodiments, the polymeric waste materials are selected from the group consisting of single plastic waste, single virgin plastic on spec or off spec, mixed plastics waste, rubber waste, cracker oil residue, used oils, biomass, and mixtures thereof. In some embodiments, the polymeric waste materials are selected from the group consisting of single plastic waste, single virgin plastic off spec, mixed plastics waste, rubber waste, and mixtures thereof. In some embodiments, the polymeric waste materials are selected from the group consisting of single virgin plastic off-spec, mixed plastics waste, and mixtures thereof.

In some embodiments, the polymeric waste material is further made from or containing limited quantities of non-pyrolysable components. In some embodiments, the non-pyrolysable components are selected from the group consisting of water, glass, stone, and metal. In some embodiments, the non-pyrolysable components are contaminants. “As used herein, the term “limited quantities” refers to an amount of less than 50 wt. %, alternatively less than 20 wt. %, alternatively less than 10 wt. %, of the total weight of the dry polymeric waste material feedstock.

In some embodiments, the polymeric waste material is extruded prior to being employed as feedstock in the process. In some embodiments, the polymeric waste material is pelletized, wherein the pellets are employed as feedstock in the process. In some embodiments, the polymeric waste material is employed in a molten state. In some embodiments, the polymeric waste material is in a molten state at temperatures from 200° C. to 300° C.

In some embodiments, the polymeric waste material employed as feedstock:
i) has a total content of volatiles (TV) of less than 5%, alternatively less than 3%, alternatively less than 2%, alternatively less than 1%, measured as the weight loss of a 10 g sample at 100° C. and a pressure of 200 mbar after 2 hours;
ii) is shredded or in pellet form;
iii) has a polyolefin content of more than 50 wt. %, alternatively more than 60 wt. %, alternatively more than 70 wt. %, alternatively more than 80 wt. %, alternatively more than 90 wt. %, based on the total weight of the polymeric waste material feedstock;
iv) has an amount of polar polymer contaminants of less than 10 wt. %, alternatively less than 5 wt. %, alternatively less than 3 wt. %, based on the total weight of the polymeric waste material;
v) has an amount of cellulose, wood, or paper of less than 10 wt. %, alternatively less than 5 wt. %, alternatively less than 3%, based on the total weight of the polymeric waste material;
vi) has a total chlorine content of less than 1.0 wt. %, alternatively less than 0.5 wt. %, alternatively less than 0.1 wt. %, based on the total weight of the polymeric waste material; or
vii) has a total ash content of less than 15 wt. %, alternatively less than 10 wt. %, alternatively less than 5 wt. %, alternatively less than 3 wt. %, determined as residue after heating the polymeric waste at 800° C. for 120 hours in air. In some embodiments, the ash content is from 0.01 to 2 wt. %, alternatively from 0.02 to 1.5 wt. %, alternatively from 0.05 to 1.0 wt. %. In some embodiments, the polymeric waste material is shredded, compacted, and has a bulk density from 70 to 500 g/l, alternatively from 100 to 450 g/l, determined according to DIN 53466. In some embodiments, the polymeric waste material is in pellet form and has a bulk density from 300 to 700 g/l, determined according to DIN 53466. In some embodiments, the polyolefin content is made from or containing polypropylene (PP), polyethylene (PE), or both.

In some embodiments, the polymeric waste materials are defined by upper limits of minor components, constituents or impurities expressed as percent by weight. In some embodiments, the lower limits for the amounts of these components, constituents or impurities are below the detection limit, alternatively are 0.001 wt. %, 0.01 wt. %, or 0.1 wt. %, respectively.

In some embodiments, the polymeric waste stream is separated using a technique selected from the group consisting of moving beds, drums and screens, and air separators, thereby differentiating materials by size, weight and density. In some embodiments, advanced sorting of plastic waste is achieved by spectroscopy techniques (MIR, NIR [near-infrared]), X-ray or fluorescence spectroscopy, thereby providing plastic waste streams with high polyolefin content.

In some embodiments, Automatic Separation Techniques of waste plastics include dry sorting technique, electrostatic sorting technique, mechanical sorting method (involves centrifugal force, specific gravity, elasticity, particle shape, selective shredding and mechanical properties), wet sorting technique, and chemical sorting methods. In some embodiments, the wet sorting technique is a sink float sorting method.

In some embodiments, the sorting techniques are described in B. Ruj et al: Sorting of plastic waste for effective recycling, Int. J. Appl. Sci. Eng. Res 4, 2015, 564-571.

### Reactor

In some embodiments, the depolymerization process is carried out in a reactor including: (a) feeding devices for introducing polymeric waste material and catalyst into the reactor; (b) a pyrolysis device equipped with heating units, gas discharge units and a solid discharge unit; and (c) a condensation device.

In some embodiments, the gas discharge units are distributed throughout the pyrolysis device and are provided with an outlet to discharge the gaseous fraction of the depolymerization and an inlet for introducing cleaning gas into the pyrolysis device.

In some embodiments, the reactor includes more than a single pyrolysis unit.

In some embodiments, the polymeric waste feedstock and the catalyst are introduced into the pyrolysis unit via a feeding device and then heated, thereby achieving depolymerization and generating gaseous fractions. In some embodiments, the polymeric waste feedstock and the catalyst are introduced via more than a single feeding device. In some embodiments, the gaseous fractions are discharged through the outlet of the gas discharge units and conveyed to the condensation unit for further processing. In some embodiments, solid residue of the depolymerization is discharged via the solid discharge unit. In some embodiments, cleaning gas for cleaning the gas discharge units and the pyrolysis unit is introduced through the inlet of the gas discharge units.

In some embodiments, the gas discharge units are equipped with filter membrane, thereby avoiding the presence of solids in the gaseous fractions after being discharged from the pyrolysis device. In some embodiments, the gas discharge units are made from or containing metallic, ceramic grain, or fiber materials.

In some embodiments, the pyrolysis device is further equipped with a screw for homogenously mixing the polymeric waste material in the pyrolysis device throughout the depolymerization. In some embodiments, the residence time of the solids in the pyrolysis device is defined by adjusting the rotational speed of the screw. In some embodiments, the pyrolysis device is operated at temperatures of 350 to 550° C.

In some embodiments, the discharged gaseous fractions of the depolymerization are conveyed to the condensation device, thereby obtaining a liquid and a gaseous depolymerization product. In some embodiments, the condensation device includes several condensers. In some embodiments, the condensers are operated at different temperatures. In some embodiments, the temperatures of the condensers are according to the boiling points of the condensates.

### Depolymerization Product

In some embodiments, the process yields a depolymerization product having selectivity. In some embodiments, the gaseous fractions generated during pyrolysis are separated into liquid and gaseous depolymerization products. In some embodiments, the separation occurs by condensation.

### Liquid Depolymerization Product

In some embodiments, the liquid depolymerization product has a low content of aromatic compounds, alternatively a low content of polycyclic aromatic compounds and asphaltanes. In some embodiments, the liquid depolymerization product has a low content of aromatic and olefinic components and a high degree of purity.

In some embodiments, the liquid depolymerization product has less than 10 mol %, alternatively less than 5 mol %, alternatively no more than 3 mol %, of aromatic components, measured as contents of aromatic protons in mol % as determined by 1H-NMR-spectroscopy.

In some embodiments, the liquid depolymerization product has a low content of olefinic compounds. In some embodiments, the liquid depolymerization product has less than 7 mol %, alternatively less than 5 mol %, alternatively less than 3 mol %, of olefinic compounds being determined based on the contents of olefinic protons, based on the total number of hydrocarbon protons as determined by 1H-NMR-spectroscopy.

In some embodiments, the content of double bonds is measured by the Bromine number (BrNo.) which indicates the degree of unsaturation. In some embodiments, the liquid depolymerization product has a Bromine number, expressed as gram bromine per 100 grams of sample, of less than 150, alternatively from 10 to 100, alternatively from 15 to 80, alternatively from 20 to 70, alternatively from 25 to 100, determined according to ASTM D1159-01.

In some embodiments, the liquid depolymerization product has a boiling range from 30 to 650° C., alternatively from 50 to 250° C. In some embodiments, the depolymerization product is separated into hydrocarbon fractionations of different boiling ranges. In some embodiments, the hydrocarbon fractions are selected from the group consisting of a light naphtha fraction made from or containing C5 and C6 hydrocarbons having a boiling range from 30° C. and 130° C., a heavy naphtha fraction made from or containing C6 to C12 hydrocarbons having a boiling range from 130° C. to 220° C., a kerosene fraction made from or containing C9 to C17 hydrocarbons having a boiling range from 220° C. to 270° C., and other high boiling point fractions selected from the group consisting of diesel fuel, fuel oil, and hydrowax. In some embodiments, the separation occurs by distillation.

In some embodiments, the liquid depolymerization product has little to no solid residue. In some embodiments, the content of residues, upon evaporation of the liquid depolymerization product, is no more than 5 ppm (w), determined according to ASTM D381.

### Gaseous Depolymerization Product

In some embodiments, the gaseous depolymerization product has a low content of low molecular hydrocarbons such as methane or ethane. In some embodiments, the gaseous depolymerization product is made from or containing high amounts of higher olefins. In some embodiments, the higher olefins are selected from the group consisting of ethylene, propylene, and butene. In some embodiments, the gaseous depolymerization product is made from or containing a high content of olefins selected from the group consisting of ethylene, propylene, and butene. In some embodiments, the gaseous depolymerization product has a low content of saturated low molecular hydrocarbons, alternatively hydrocarbons of the formula CnH2n+2 wherein n is a real number ranging from 1 to 4.

In some embodiments and after step (e) of the depolymerization process, the gaseous depolymerization product has a content of methane of at most 6 wt. %, alternatively at most 4 wt. %, alternatively at most 3 wt. %, alternatively at most 2 wt. %, alternatively at most 0.5-1.5 wt. %, based on the total weight of the gaseous depolymerization product.

In some embodiments, the gaseous depolymerization product is made from or containing a high amount of low molecular olefinic compounds. In some embodiments, the low molecular olefinic compounds have the formula CnH2n with n=2-4. In some embodiments, the gaseous fraction is used directly as feedstock for further processing in a cracker downstream, thereby obtaining purified monomer streams, and thereafter for the subsequent production of polymers, thereby bypassing the energy-consuming stream cracking ovens and reducing the output of CO2. In some embodiments, the downstream cracker is a raw gas compressor. In some embodiments and after step (e), the gaseous depolymerization product has at least 50 wt. %, alternatively at least 60 wt. %, alternatively at least 65 wt. %, alternatively at least 70 wt. %, alternatively at least 75 wt. %, of a content of compounds of the formula CnH2n (olefins) with n=2-4, based on the total weight of the gaseous depolymerization product.

In some embodiments, the gaseous depolymerization product has small quantities of HCl, HCN, H2S, H2O, NH3, or COS. In some embodiments, the quantities of HCl, HCN, H2S, H2O, NH3, and COS are separated in a refining step before introduction to the steam cracker downstream segments.

The present disclosure will be explained in more detail with reference to the figures and the examples provided below. While the figures illustrate various implementations described herein, the accompanying figures do not limit the scope of the present disclosure.

### FIGURES

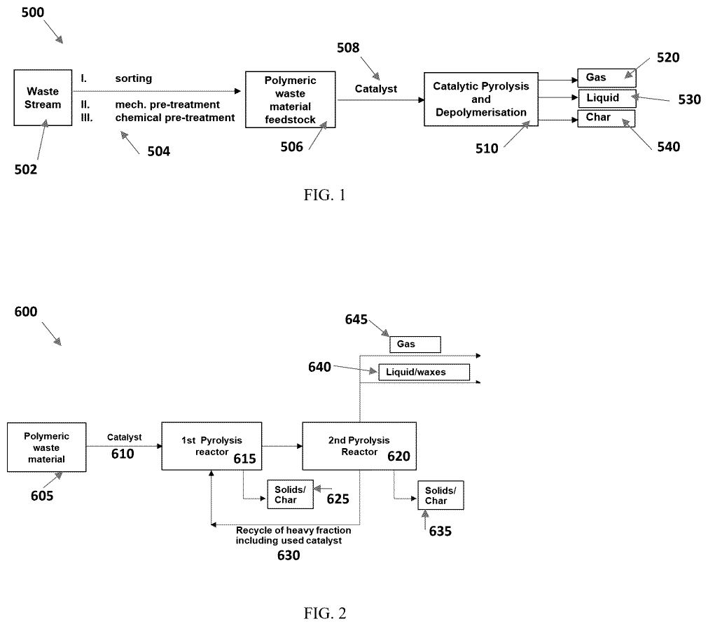

FIG. 1 is a flowchart, depicting a process for depolymerization of plastic waste material. In the process 500, a waste stream 502 is sorted to separate polymeric waste material from other materials and optionally, mechanically or chemically pre-treated 504, thereby producing polymeric waste material feedstock 506. Polymeric waste material feedstock 506 is then mixed with catalyst 508 and subjected to catalytic pyrolysis, thereby depolymerizing 510 the polymeric materials. Depolymerization of the polymeric material generates gaseous products 520, liquid products 530, and solid products 540. The products are separated. In some instances, the gaseous and liquid products 520, 530 are further processed. In some instances, solid product 540 is further processed, thereby separating char from the catalyst 508. In some instances, the recovered catalyst is used in a subsequent iteration of the process.

FIG. 2 is a flowchart, depicting a process for depolymerization of plastic waste material. In process 600, polymeric waste material 605 is mixed with catalyst 610, thereby forming a mixture, and sent to first pyrolysis reactor 615. In first pyrolysis reaction 615, the mixture is heated, thereby depolymerizing at least a portion of the polymeric waste material. After removing certain solids and char 625 produced in the first pyrolysis reactor, the remaining mixture is transferred to second pyrolysis reactor 620. In second pyrolysis reactor 620, the remaining mixture is again heated, thereby depolymerizing the remaining polymeric waste material. In some instances, gases 645 produced during depolymerization are sent to steam cracker (not shown) while liquids and waxes 640 are directed to further processing.

### Examples

The following analytical methods were employed:

1) GC MS was used for liquid and gas analysis.

2) Char residue was determined according to mass balance after decoking the residues of the reactor at 800° C.

3) Liquid contents were characterized using simulated distillation (SimDist) analysis according to ASTM D 7213: 2012. Final boiling point (FBP), boiling temperature at 50% and initial boiling point (IBP) were taken from SimDist.

4) The total content of unsaturated components in the liquid condensates were characterized via Bromine number determination using an 848 Titrino Plus (Metrohm AG, Herisau, Switzerland), equipped with a double PT-wire electrode, which had integrated PT1000 temperature sensor, and a 10 ml burette, in accordance with ASTM D1159-01, as described in Metrohm Application Bulletin 177/5e, December 2018. The Bromine number (BrNo.) represented the amount of bromine in grams absorbed by 100 grams of a sample.

5) 1H-NMR analysis was conducted by dissolving a sample of the liquid condensate in CDCl3 and characterizing the sample using proton NMR spectroscopy. Aromatic, olefinic and aliphatic protons were assigned according to the chemical shifts summarized in Table 1:

TABLE 1
Integral Regions in 1H-NMR spectroscopy

| Peak Assignment | 1H Chemical Shift (ppm) |
|---|---|
| I₁ (Aromatic Protons) | 8.25-7.27 |
| CDCl₃ -Solvent | 7.26 |
| I₂ (Aromatic Protons) | 7.25-6.60 |
| I₃ (Olefinic Protons - Type 2) | 6.60-5.95 |
| I₄ (Olefinic Protons - Type 1) | 5.95-5.67 |
| I₅ (Olefinic Protons - Type 2) | 5.67-5.35 |
| I₆ (Olefinic Protons - Type 3) | 5.35-5.15 |
| I₇ (Olefinic Protons - Type 1) | 5.15-4.85 |
| I₈ (Olefinic Protons - Type 4) | 4.85-4.40 |
| I₉ . . . (Paraffinic Protons) | 4.40-0.25 |

The listed types of olefinic protons were assumed to correspond to the following structures:

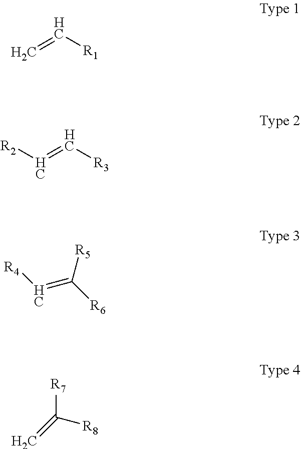

The amount of aromatic, olefinic and aliphatic protons were determined based on the assigned peak integrals according to the following equations:

$$Mol \% Aromatic Protons = [ \text{} ( {I}_{1} + {I}_{2} ) / ( {I}_{1} + {I}_{2} + {I}_{3} + {I}_{4} + {I}_{5} + {I}_{6} + {I}_{7} + {I}_{8} + {I}_{9} ) ] \%$$

$$Mol \% Olefinic Protons Type 1 = [ \text{} ( {I}_{4} + {I}_{7} ) / ( {I}_{1} + {I}_{2} + {I}_{3} + {I}_{4} + {I}_{5} + {I}_{6} + {I}_{7} + {I}_{8} + {I}_{9} ) ] \%$$

$$Mol \% Olefinic Protons Type 2 = [ \text{} ( {I}_{3} + {I}_{5} ) / ( {I}_{1} + {I}_{2} + {I}_{3} + {I}_{4} + {I}_{5} + {I}_{6} + {I}_{7} + {I}_{8} + {I}_{9} ) ] \%$$

$$Mol \% Olefinic Protons Type 3 = [ \text{} ( {I}_{6} ) / ( {I}_{1} + {I}_{2} + {I}_{3} + {I}_{4} + {I}_{5} + {I}_{6} + {I}_{7} + {I}_{8} + {I}_{9} ) ] \%$$

$$Mol \% Olefinic Protons Type 4 = [ \text{} ( {I}_{8} ) / ( {I}_{1} + {I}_{2} + {I}_{3} + {I}_{4} + {I}_{5} + {I}_{6} + {I}_{7} + {I}_{8} + {I}_{9} ) ] \%$$

$$Mol \% Paraffinic Protons = [ \text{} ( {I}_{9} ) / ( {I}_{1} + {I}_{2} + {I}_{3} + {I}_{4} + {I}_{5} + {I}_{6} + {I}_{7} + {I}_{8} + {I}_{9} ) ] \%$$

6) The water content of the catalyst was determined using a Sartorius MA45 (Sartorius AG, Goettingen, Germany) on a sample of 0.5 to 1 g at 180° C.

7) For the determination of a pH value of the hydrodepolymerization products, a liquid sample of the hydrodepolymerization product was extracted with water in a volume ratio water:sample of 1:5. The pH value of the aqueous solution was measured.

8) Particle size distribution of the particulate non-porous support and the catalyst were determined according to Coulter counter analysis in accordance with ASTM D4438.

9) Properties of the organic waste material feedstock were determined as follows:

Samples of from 20 to 100 g of the polymeric waste were milled and analyzed. Alternatively, a pelletized sample of the polymeric waste was analyzed. The following methods were used:
i) Total Volatiles (TV) were measured as the weight loss of a 10 g sample at 100° C. and after 2 hours at 200 mbar.
ii) Water content was determined by Karl-Fischer titration, using an apparatus from Metrohm 915 KF Ti-Touch equipped with a PT100 indicator electrode for volumetric KF titration, according to Metrohm Application Bulletin 77/3e in compliance with ASTM E203.
iii) IR-Spectroscopy was used for a qualitative identification of various polymers (PP, PE, PS, PA, PET, PU, and Polyester) and additives such as CaCO3.
iv) Standard elemental analysis was used for determination of wt. % of H, C, N (DIN 51732: 2014-07) and S (tube furnace, ELTRA GmbH, Haan, Germany, DIN 51724-3: 2012-07).
v) 1H-NMR was used for determining the composition of polymers soluble in solvents for recording a 1H-NMR spectrum: PE/PP balance (copolymers were also included), PET, PS.
vi) Ash Content analysis of plastics was determined at 800° C. according to DIN EN ISO 3451-1 (2019-05).
vii) Bulk density of the polymer waste was determined according to DIN 53466.
viii) Corrosivity was determined as the pH value of an aqueous solution after a contact time of 3 h (5 g sample in 50 ml distilled water).
ix) Inductively coupled plasma atomic emission spectroscopy (ICP-AES) was used for quantitative element determination (total chlorine content, content of Si or metals).
x) The ash content of a liquid feedstock such as pyrolysis oil, was measured according to ASTM D482-19.

Various catalysts were prepared and tested in depolymerizations of different polymeric waste materials. The particulate non-porous support used in the catalysts was sand, having a particle size distribution, as summarized in Table 2, with 99% of the particles being smaller than 3 mm. Prior to use in formation of the catalyst, the sand was pre-dried at 80° C. for 24 h in a drying oven with circulating air.

TABLE 2
Particle size distribution

| | Sand, wt. % |
|---|---|
| >1400 μm | 40.55% |
| >1400 μm->1000 μm | 55.30% |
| <1000 μm | 4.15% |

### Comparative Examples

Comparative catalysts were formed by mixing an acidic compound with 25 kg of the sand, thereby obtaining the comparative catalysts (Comp-1, Comp-2, and Comp-3) identified in Table 4.

### Examples

Catalysts using a mineral oil coating agent were prepared as follows:

25.0 kg of sand was placed into a 60 L steel barrel with screw cap and equipped with a Teflon inlay. 350 ml mineral oil were added (corresponding to 1.4 wt % with respect to sand). The drum was placed on a Drum Hoop Mixer and rotated for 1 hour (about 100 rpm). 24.5 kg of the resulting mixture was placed in another drum. 500 g of the acidic component (corresponding to a 2 wt % loading) were added. The drum was placed on a drum hoop mixer and rotated for 1 hour (about 100 rpm). At the end of the mixing process, a free-flowing catalyst was obtained with an even distribution of the particles of the acidic component on the surface of the sand particles.

The mineral oil used was Ondina X 432, which was commercially available from Shell, having the following properties:

TABLE 3

| Property | Method | Shell Ondina X 432 |
|---|---|---|
| Kinematic Viscosity at | ISO 3104 | 165 |
| 20° C. in mm²/s | | |
| Kinematic Viscosity at | ISO 3104 | 59 |
| 40° C. in mm²/s | | |
| Kinematic Viscosity at | ISO 3104 | 9.0 |
| 100° C. in mm²/s | | |

Catalysts using a silica hydrogel coating agent were prepared as follows:

25.0 kg of sand were placed into a 60 L steel barrel with screw cap and equipped with a Teflon inlay. 500 ml water were added (corresponding to 2.0 wt % with respect to sand). The drum was placed on a Drum Hoop Mixer and rotated for 1 hour (about 100 rpm). 24.5 kg of the resulting mixture was placed in another drum. 1000 g of a 1:1 milled free-flowing mixture of silica hydrogel and the acidic compound (corresponding to a 2 wt % loading) were added. The drum was placed on a drum hoop mixer and rotated for 1 hour (about 100 rpm). At the end of the mixing process, a free-flowing catalyst was obtained with an even distribution of the particles of the acidic compound on the surface of the sand particles. The resulting mixture was dried at 120° C. vacuum for 6 h.

The silica hydrogel was prepared, according to the procedure described in European Patent Application No. EP1290042, example 1. The solid content of the hydrogel sample was 20 wt %. The D50 of the milled mixture of silica hydrogel and acidic components were between 80-100 μm in accordance with ASTM D4438.

Table 4 summarizes the catalysts employed with the amount of the acidic compound given in wt. % with respect to sand.

TABLE 4

| | | | Amount Acidic |
|---|---|---|---|
| Catalyst # | Coating Agent | Acidic Compound | Compound (wt. %) |
| 1 | oil | Siral 40 HPV | 1 |
| 2 | oil | Siral 40 HPV | 2 |
| 3 | oil | Zeolite CFG-1 | 1 |
| 4 | silica hydrogel | Siral 40 HPV | 1 |
| 5 | silica hydrogel | Siral 40 HPV | 2 |
| 6 | silica hydrogel | Zeolyst Beta | 2 |
| 7 | silica hydrogel | Zeolyst ZSM-5 | 2 |
| 8 | oil | Zeolyst Beta | 2 |
| 9 | oil | Zeolyst ZSM5 | 2 |
| Comp-1 | none | Zeolite CFG-1 | 2 |
| Comp-2 | none | Siral 40 HPV | 2 |
| Comp-3 | none | Zeolyst Beta | 2 |
| Comp-4 | none | Zeolyst ZSM5 | 2 |

### Acidic Compounds:

Zeolite CFG-1, Zeolyst ZSM-5 and Zeolyst Beta (CP811E-75) were commercially available from PQ Corporation, Malvern, PA, USA

Siral 40 HPV: Si/Al mixed oxide with Al2O3/SiO2 60/40 [%], was commercially available from Sasol Germany GmbH, Hamburg, Germany.

### Feedstock:

Some of the following organic waste materials were employed as feedstocks:
A: Pelletized agricultural and industrial packaging film.
B: Shredded flakes and small pieces of film.
C: Flakes, fluff and small pieces of film, compacted.
D: Shredded film.
E: Shredded and pelletized multilayer film.
G) 50/50 PE/PP mixture virgin polymer in pellets.
H) End-of-life vehicle shredded plastic waste fraction with metal impurities.

The properties of the feedstocks, averaged on analysis of three samples, are summarized in Table 5.

TABLE 5

| | | | | | | Polymer composition analysis | | | | | |
|---|---|---|---|---|---|---|---|---|---|---|---|
| | | | | | | | | | | | Other |
| Feed- | Ash | TV | BD | Cl | PE + PP | PE | PP | PET | PS | PA | cont.* |
| stock | wt % | wt % | g/cm³ | wt % | wt % | wt % | wt % | wt % | wt % | wt % | wt % |
| A | 1.5 | 0.1 | 494 | 0.01 | 95 | 83 | 12 | <0.1 | 1.7 | 0.7 | 2.6 |
| B | 1.6 | 1.8 | 180 | 0.06 | 92 | 41 | 51 | 1.3 | 0.8 | 0.5 | 5.4 |
| C | 3.8 | 1.8 | 250 | 0.06 | 90 | 51 | 39 | 0.2 | 1.8 | 2.2 | 5.8 |
| D | 0.5 | 3.5 | 55 | 0.09 | 93 | 53 | 40 | <0.1 | 1 | — | 6.0 |
| E | 0.4 | 0.2 | 530 | <0.01 | 72 | 20 | 52 | 12 | 0 | 10 | 6 |
| G | <0.1 | 0.1 | 525 | <0.01 | 100 | 50 | 50 | — | — | — | — |
| H | 28 | 3.5 | 350 | 0.2 | 55% | 20 | 35 | — | — | — | 45 |

*Other content includes inorganic, polymer or organic contaminants and volatile components
Ash: ash content
TV: total volatiles
BD: bulk density
Cl: total chlorine content
PE: polyethylene content
PP: polypropylene content
PET: content of polyethylene terephthalate
PS: polystyrene content
PA: polyamide content
Other cont.: content of other contaminants

The feedstock and catalyst were introduced into a reactor device equipped with heating units, gas discharge units, solid discharge unit; a condensation device; and a screw for homogenously mixing the reactor content during depolymerization. Conditions of the depolymerization conducted are summarized in Table 6. The resulting gaseous fractions were further separated into liquid and gaseous depolymerization products by condensation. The amounts of the resulting fractions are also given in Table 6.

TABLE 6
Process parameter and mass balance ^{(a)}

| Run# | Temp | Time | Cat# | Feedstock | % Wax**⁾ | % Liquid | % Gas |
|---|---|---|---|---|---|---|---|
| 1 | 500° C. | 30 min | none | G | 44.7 | 0 | 51.7 |
| 2 | 500° C. | 30 min | Comp-1 | G | 0* | 37.4* | 58.7 |
| 3 | 500° C. | 30 min | Comp-2 | G | 0* | 43.4* | 54.5 |
| 4 | 500° C. | 30 min | Comp-3 | G | 0* | 58.3* | 36.5 |
| 5 | 450° C. | 30 min | Comp-1 | G | 54.4* | 0* | 32.1 |
| 6 | 500° C. | 30 min | 1 | G | 0 | 61.8 | 37.1 |
| 7 | 450° C. | 30 min | 1 | G | 0 | 64.7 | 26.6 |
| 8 | 450° C. | 30 min | 2 | G | 0 | 58.8 | 31.8 |
| 9 | 450° C. | 30 min | 3 | G | 0 | 63.8 | 24.0 |
| 10 | 450° C. | 30 min | Comp -2 | G | 56.4 | — | 32.1 |

^{(a)} Amount missing to 100% is due to losses
*high amount of waxes floating in an inhomogeneous liquid
**% Wax: a solid product at room temperature

With reference to the results of Table 6, comparative runs #2-5 showed an unstable depolymerization with peaks in gas generation corresponding to a black/brown liquid with wax floating. As such, it is believed that both thermal pyrolysis and catalytic depolymerization were taking place at the same time. Notwithstanding, run #5 compared to run #2, was performed at 50° C. lower and delivered much higher amounts of waxes.

Catalysts 1, 2 and 3 which were provided with the coating agent, thereby enabling a smooth run (Run #6-9) with constant gas generation at lower temperature and yielded a homogenous clear yellow-brown liquid.

The liquid and gaseous depolymerization products were further analyzed. The results of the analysis of the liquid depolymerization product are summarized in Table 7.

TABLE 7
Composition liquid depolymerization product

| | Liquid | | | | | | | | |
|---|---|---|---|---|---|---|---|---|---|
| | Fraction | | | | Br- | | | | |
| | (Condensing | % mol | % mol | % mol | Number | Sulfur | IBP* | 50%* BP | FBP* |
| Run# | Unit) | Arom⁽¹⁾. | Olefin | Aliph. | [g/100 g] | [mg/kg] | [° C.] | [° C.] | [° C.] |
| 6 | C1/C2 Mix | 1.4 | 4.14 | 94.46 | 68.7 | 4.6 | 72.9 | 237.8 | 563.9 |
| 7 | C1 | 1.06 | 3.91 | 95.03 | 70.2 | <3 | 36.1 | 244.2 | 532.3 |
| 7 | C2 | 1.06 | 4.37 | 94.57 | 78.2 | 3.6 | 36.2 | 217.0 | 549.2 |
| 8 | C2 | 1.08 | 3.59 | 95.33 | 46.6 | <3 | 59.5 | 231.6 | 576.4 |
| 8 | C1 | 0.98 | 3.15 | 95.88 | 36.4 | <3 | 71.8 | 268.5 | 583.2 |

1- HNMR has been used to obtain the Mol % of aromatic, olefinic and aliphatic protons in the sample
*Final boiling point (FBP), boiling temperature at 50% and initial boiling point (IBP) from SimDist.

The depolymerization of polymeric waste material with the catalyst yielded a high amount of liquefiable product, which contained small contents of aromatic and olefinic content.

In another set of tests, a different feedstock was depolymerized, thereby showing that, for a given feedstock, the catalyst produces an increased portion of gaseous fractions in the depolymerization reaction with respect to pure thermal depolymerization (Run 11) and comparative catalysts. The process parameter and mass balance of the resulting depolymerization product are summarized in Table 8.

TABLE 8
Process parameter and mass balance ^{(a)}

| Run# | Temp | Time | Cat# | Feedstock | % Wax | % Liquid⁺⁾ | % H₂O | % Gas | % Residue |
|---|---|---|---|---|---|---|---|---|---|
| 11 | 450° C. | 30 min | none | A | 66.5 | 0 | 0.7 | 31.0 | 1.8 |
| 12 | 450° C. | 30 min | 8 | A | 0 | 15.0 | 0.5 | 83.0 | 1.5 |
| 13 | 450° C. | 30 min | 8 | A + 1% | 0 | 26.7 | 1.2 | 71.1 | 1.0 |
| | | | | PA | | | | | |
| 14 | 450° C. | 30 min | 8 | A + 5% | 0 | 23.5 | 2.3 | 73.2 | 1.0 |
| | | | | paper | | | | | |
| 15 | 450° C. | 30 min | 8 | A + 10% | 0 | 65.4 | 1.5 | 31.7 | 1.4 |
| | | | | PA | | | | | |
| 16 | 450° C. | 30 min | 9 | A | 0 | 26.0 | 2.0 | 69.0 | 3.0 |
| 17 | 450° C. | 30 min | 7 | A | 0 | 15.4 | 2.8 | 80.4 | 1.4 |
| 18 | 450° C. | 30 min | 6 | A | 0 | 19.9 | 0.4 | 76.8 | 2.6 |
| 19 | 450° C. | 30 min | 7*) | A | 0 | 16.9 | 0.1 | 82.9 | 0.1 |
| 20 | 450° C. | 30 min | 8 | B | 0 | 36.2 | 1.0 | 52.9 | 8.9 |
| 21 | 450° C. | 30 min | 8 | C | 0 | 59.9 | 0.5 | 27.2 | 9.2 |
| 22 | 450° C. | 30 min | Comp-3 | A | 62.3^{b)} | 0 | 1.2 | 26.8 | 8.6 |

| | | | | | |
|---|---|---|---|---|---|
| 23 | 450° C. | 30 min | Comp-3 | B | The run failed due to |
| | | | | | agglomerates and reactor fouling |

^{(a)} Amount missing to 100% is due to losses;
^{b)}high amount of waxes floating in an inhomogeneous liquid
⁺⁾Liquid was the combination of the fractions, may have contained waxy solid particles and agglomerates which disappeared upon heating to >50° C.
*Catalyst, which was used in three successive runs, maintained good selectivity and then regenerated by heating up to 800° C. in air and sieving out the smaller particles <500 μm (ashes).

The above data show that the catalysts produce high amounts of gaseous depolymerization products and maintain good performances even when the feedstock is added with heterogeneous material. Moreover, the catalyst can be regenerated, thereby allowing the possibility to increase process sustainability.

The results of the analysis of the gaseous depolymerization product are summarized in Table 9.

TABLE 9
mass balance of the gaseous depolymerization product

| | Run# | | | | | | | |
|---|---|---|---|---|---|---|---|---|
| | 11 | 12 | 13 | 14 | 15 | 17 | 18 | 19 |
| | Feedstock | | | | | | | |
| | A | A | A + 1% PA | A + 5% paper | A + 10% PA | A | A | A |
| | Catalyst # | | | | | | | |
| | none | 8 | 8 | 8 | 8 | 7 | 6 | 7*⁾ |
| Components | Wt. % | Wt. % | Wt. % | Wt. % | Wt. % | Wt. % | Wt. % | Wt. % |
| H₂ | 0.1 | 0 | 0 | 0 | 0 | 0.1 | 0.1 | 0.2 |
| CO | 1.1 | 1.8 | 2 | 3.2 | 2.7 | 0 | 0 | 0 |
| CO₂ | 4 | 11.2 | 11.5 | 13.9 | 11.8 | 0.4 | 2.4 | 0.6 |
| CH₄ | 6.1 | 0.5 | 0.9 | 0.7 | 5.6 | 0 | 0 | 0.2 |
| C₂H₆ | 13.5 | 0.4 | 1 | 0.6 | 10.8 | 0.8 | 0.3 | 1.1 |
| C₂H₄ | 11.1 | 0.9 | 1.3 | 1 | 10.2 | 5.4 | 0.8 | 5.8 |
| C₃H₈ | 14.8 | 2.1 | 2.1 | 1.9 | 11.5 | 13.3 | 1.3 | 16.6 |
| C₃H₆ | 20.3 | 27.9 | 27.7 | 25.9 | 18.9 | 29.2 | 33.3 | 25.5 |
| Butanes | 11.9 | 23.3 | 20 | 19.6 | 9.7 | 24.7 | 21.5 | 23 |
| Butenes | 16.3 | 32 | 33.5 | 33.2 | 18.7 | 26 | 40.4 | 26.9 |
| Rest | 0.7 | 0 | 0 | 0 | 0 | 0 | 0 | 0 |
| Olefins | 47.7 | 60.8 | 62.5 | 60.1 | 47.8 | 60.6 | 74.5 | 58.2 |
| total HC | 94 | 87.1 | 86.5 | 82.9 | 85.4 | 99.4 | 97.6 | 99.1 |
| Olefins/HC | 50.7 | 69.8 | 72.3 | 72.5 | 56.0 | 61.0 | 76.3 | 58.7 |

*Catalyst, which was used in three successive runs, maintained good selectivity and then regenerated by heating up to 800° C. in air and sieving out the smaller particles <500 μm (ashes)
HC: Hydrocarbons
Olefins: sum of ethylene, propylene and butenes
Olefins/HC: percentage of the olefins over total hydrocarbons

As can be seen from Table 9, the process and the catalyst yielded gaseous depolymerization products with high amounts of monomers useful as feedstock for polymerization after purification. In contrast thereto, thermal pyrolysis, as reflected by run #11, did not show selectivity towards olefins and yielded high amounts of methane.

1. A catalyst for depolymerization of polymeric waste material comprising, as an active component, an acidic compound which is deposited on a particulate non-porous support with the aid of a coating agent.

2. The catalyst of claim 1, wherein the particulate support is selected from the group consisting of sand, glass beads and metal particles.

3. The catalyst of claim 2, wherein the support has an average particle size D50 of 0.2 to 20 mm, determined according to sieve analysis in accordance with ISO 3310-1/ASTM E11.

4. The catalyst of claim 3, wherein the support is sand.

5. The catalyst according to claim 1, wherein the acidic compound is selected from the group consisting of Al/Si mixed oxides, Al₂O₃, aluminosilicates, silica and zeolites.

6. The catalyst of claim 1, comprising 0.5 to 6 wt. % of the active component, based on the total weight of the catalyst.

7. The catalyst of claim 1, wherein the coating agent is selected from the group consisting of oil, inorganic hydrogel, and combinations thereof.

8. The catalyst of claim 1, comprising from 1 to 300% wt of the coating agent, based on the amount of acidic compound.

9. A process for the depolymerization of polymeric waste material, comprising the steps of:
(a) providing a feedstock of polymeric waste material;
(b) mixing the feedstock with a catalyst of claim 1, thereby forming a mixture;
(c) pyrolyzing the mixture, thereby generating gaseous fractions;
(d) collecting the gaseous fractions; and
(e) separating the collected gaseous fraction, thereby obtaining a gaseous and a liquid depolymerization product.

10. The process of claim 9, further comprising the step of (f) distilling the liquid depolymerization product.

11. The process of claim 9, wherein the polymeric waste material comprises a plastic material selected from the group consisting of polyethylene (PE), polypropylene (PP), polystyrene (PS), polyethylene terephthalate (PET), polyvinylchloride (PVC), polyamide (PA), polyurethane (PU), polyacrylonitrile (PAN), polybutylene (PB), and mixtures thereof.

12. The process of claim 9, wherein, after step (e), the depolymerization product comprises more than 30 wt.-% of the gaseous content, based on the combined weight of the gaseous and liquid depolymerization product.

13. The process of claim 9, wherein, after step (e), the gaseous depolymerization product comprises at least 50 wt. % of compounds of the formula CₙH₂ₙ with n=2-4, based on the total weight of the gaseous depolymerization product.

14. The process of claim 9, wherein the depolymerization catalysts is separated from solid content remaining after the depolymerization process and reintroduced into the process.

15. The process according to claim 14, wherein the catalyst is reactivated by heating before being reintroduced into the process.
